(19) United States
(12) Patent Application Publication
(10) Pub. No.: US 2025/0009382 A1
(43) Pub. Date: Jan. 9, 2025

(54) DEVICES, SYSTEMS, AND METHODS FOR NEEDLE ACCESS

(71) Applicant: Boston Scientific Scimed Inc., Maple Grove, MN (US)

(72) Inventors: Kian S. LIM, Shrewsbury, MA (US); Niraj Prasad RAUNIYAR, Plymouth, MN (US)

(73) Assignee: Boston Scientific Scimed Inc., Maple Grove, MN (US)

(21) Appl. No.: 18/763,100

(22) Filed: Jul. 3, 2024

Related U.S. Application Data
(60) Provisional application No. 63/512,087, filed on Jul. 6, 2023.

(51) Int. Cl. A61B 17/34; A61B 6/12; A61B 90/00

(52) U.S. Cl. CPC A61B 17/3403; A61B 6/12; A61B 90/39; A61B 2090/3966

(57) ABSTRACT

Medical devices and related methods useful for needle access during medical procedures are described. The medical device may include a needle and a handle coupled to the needle, wherein the handle includes a body comprising a radiolucent material, a first marker proximate a proximal end of the body and a second marker proximate a distal end of the body. Each of the first marker and the second marker may include a radiopaque material, and the first marker may have a shape complementary to the second marker to align a position of the needle relative to a subject when visualized by X-ray imaging.

### CROSS-REFERENCE TO RELATED APPLICATIONS

This application claims the benefit of priority to U.S. Provisional Application No. 63/512,087, filed on Jul. 6, 2023, which is incorporated by reference herein in its entirety.

### TECHNICAL FIELD

Aspects of the present disclosure generally relate to medical devices, systems, and procedures related thereto. In particular, some aspects relate to medical systems, devices, and methods for needle access to the urinary tract for removal of a stone.

### BACKGROUND

Minimally-invasive and non-invasive surgical procedures allow a medical professional to treat an internal area of a body through a natural body orifice or minimizing an incision of the body. Percutaneous nephrolithotomy (PCNL), for example, is a procedure wherein a needle is inserted through the skin and into a kidney for removal of a kidney stone. Precise placement of the needle aided by medical imaging techniques is required to avoid damaging the kidney or surrounding tissues. PCNL procedures often necessitate multiple medical professionals having significant experience not only with anatomy in and around the kidney but also with imaging to ensure the accurate location and angle of access of the access needle. For example, in the Bulls Eye Technique, a C-arm with an X-ray generator is used to determine angle of access for PCNL. The C-arm is rotated between 0 degrees and 30 degrees to align the X-ray generator and check the depth at which the needle is inserted into the patient.

### SUMMARY

The present disclosure includes medical devices and related methods useful for needle access during medical procedures. For example, the present disclosure includes a medical device comprising a needle and a handle coupled to the needle, wherein the handle includes a body comprising a radiolucent material, a first marker proximate a proximal end of the body and a second marker proximate a distal end of the body, each of the first marker and the second marker comprising a radiopaque material, wherein the first marker has a shape complementary to the second marker to align a position of the needle relative to a subject when visualized by X-ray imaging. The needle may include a plurality of measurement marks distributed along a length of the needle. In some examples, the needle is at least 10 cm in length. According to some aspects, the first marker and the second marker may be visible from a proximal-most end of the handle through the radiolucent material. Additionally or alternatively, the first marker and the second marker may be oriented perpendicular to a longitudinal axis defined by the needle. In some examples, one of the first marker or the second marker is a shaded shape and the other of the first marker or the second marker is a perimeter of the shaded shape. The radiolucent material of the body of the handle may comprise glass or a polymer. Optionally, the handle may include a hub at a distalmost end of the body, e.g., between the second marker and the needle.

Also disclosed herein is a medical system comprising a medical device as described above and/or elsewhere herein. For example, the medical system may further comprise a camera configured to observe the needle of the medical device and/or a fixture configured to receive the needle and maintain a position of the needle relative to the fixture during a medical procedure. According to some aspects, the fixture includes the camera. The medical device and/or medical system may be used to remove a stone from a urinary tract of a subject, e.g., during a PCNL procedure. For example, the needle may access a kidney of the subject for removal of the stone. During the use, the first marker and the second marker of the medical device may be aligned with each other along the longitudinal axis by X-ray imaging before the needle accesses the kidney. Further, for example, a depth of the needle inserted into the subject may be determined according to a plurality of measurement marks on the needle.

The present disclosure also includes a medical system comprising: a medical device comprising a needle including a plurality of measurement marks distributed along a length of the needle; and a handle coupled to the needle, wherein the handle includes a body comprising a radiolucent material, a first marker proximate a proximal end of the body, and a second marker proximate a distal end of the body, each of the first marker and the second marker comprising a radiopaque material; and the system further comprising a fixture configured to receive the needle and maintain a position of the needle relative to a subject during a medical procedure. The first marker may have a shape complementary to the second marker to align a position of the needle relative to the fixture and relative to the subject when visualized by X-ray imaging. The first marker and the second marker may be visible from a proximal-most end of the handle through the radiolucent material. Further, for example, the fixture may include a camera configured to observe the needle during the medical procedure. According to some aspects, the radiolucent material of the body of the handle may comprise glass or a polymer. In some examples, the handle includes a hub at a distalmost end of the body, the hub being between the second marker and the needle.

The present disclosure also includes a method of treating a subject, the method comprising: adjusting an orientation of a medical device relative to a back of the subject, wherein the medical device comprises: a needle and a handle coupled to the needle, wherein the handle includes a body comprising a radiolucent material, a first marker proximate a proximal end of the body and a second marker proximate a distal end of the body, each of the first marker and the second marker comprising a radiopaque material; the method further comprising inserting the needle into the subject to access a kidney of the subject; wherein adjusting the orientation of the medical device includes aligning the first marker with the second marker by X-ray imaging. The needle may be inserted while the needle forms an angle between about 25° and about 35° relative to the back of the subject. The radiolucent material of the medical device may comprise glass or a polymer, for example. The needle may be inserted through an opening of a fixture secured to the back of the subject, wherein the fixture maintains the orientation of the medical device relative to the subject. According to some aspects of the present disclosure, the fixture includes a camera, and the method further comprises monitoring insertion of the needle with the camera.

### BRIEF DESCRIPTION OF THE FIGURES

The accompanying drawings, which are incorporated in and constitute a part of this application, illustrate exemplary aspects that, together with the written descriptions, serve to explain the principles of this disclosure.

FIG. 1 illustrates an exemplary medical system used for a PCNL procedure.

FIG. 2 illustrates an exemplary medical device in accordance with some aspects of the present disclosure.

FIGS. 3A-3C illustrate alignment of exemplary markers of a medical device, in accordance with some aspects of the present disclosure.

FIGS. 4A-4C illustrate additional exemplary markers of medical devices, in accordance with some aspects of the present disclosure.

FIG. 5 illustrates an exemplary medical system in accordance with some aspects of the present disclosure.

### DETAILED DESCRIPTION

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device. In contrast, “distal” refers to a position relatively further away from the medical professional using the medical device, or closer to the interior of the body. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within ±10% of a stated value.

While aspects of the present disclosure are described in reference to PCNL procedures, the disclosure is not so limited. Reference to a particular type of medical procedure and medical device are provided for illustration purposes and not intended to limit the present disclosure. Accordingly, the exemplary systems, devices, and methods described herein may be utilized for or with other appropriate procedures, medical devices, areas of treatment, and imaging technology. For example, other energy emitting devices similar to fluoroscopes may be suitable for use with devices and methods according to the present disclosure. Additionally, although some of the examples described herein refer to radiopaque materials for use in fluoroscopy, other imaging systems such as ultrasound, MRI, or CAT-scan devices, for example, may be used in conjunction with the present disclosure.

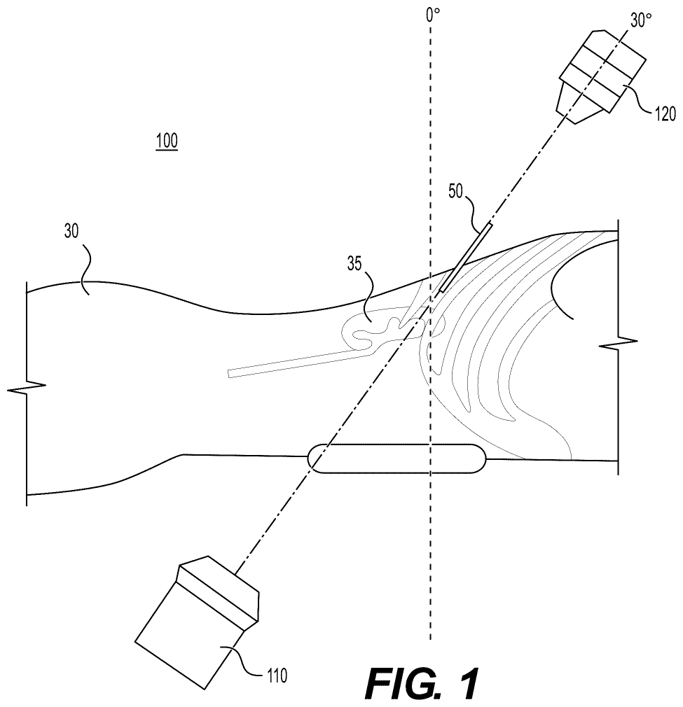

FIG. 1 shows an exemplary medical system 100 used for a PCNL procedure for a subject 30. PCNL typically includes making a relatively small (e.g., 1 cm) incision in the subject's lower back, wherein a needle is used to access the target site within the kidney for removal of a kidney stone. The medical system 100 includes a medical device comprising a needle 50 and an imaging device that includes a C-arm machine including an X-ray generator 110 and an image intensifier 120 to guide the position of the needle 50 relative to the subject 30 under X-ray imaging. The X-ray generator 110 is aligned with the image intensifier 120 so that a beam of electromagnetic energy, e.g., an X-ray beam, is received by image intensifier 120. During a typical PCNL procedure, the C-arm machine may begin in a 0-degree position, so that the X-ray beam is perpendicular with the horizon (e.g., perpendicular to the subject in a prone position). The C-arm machine may then be rotated into a 30-degree position as shown in FIG. 1 to access a calyx of the kidney 35 and minimize the risk of damaging surrounding tissues. A medical professional may rotate the C-arm machine between the 0-degree position to determine an appropriate depth of insertion of the needle 50 to access the kidney 35, and the 30-degree position for the appropriate angle of entry. Rotating the C-arm machine can present difficulties that may be reduced or eliminated by aspects of the present disclosure.

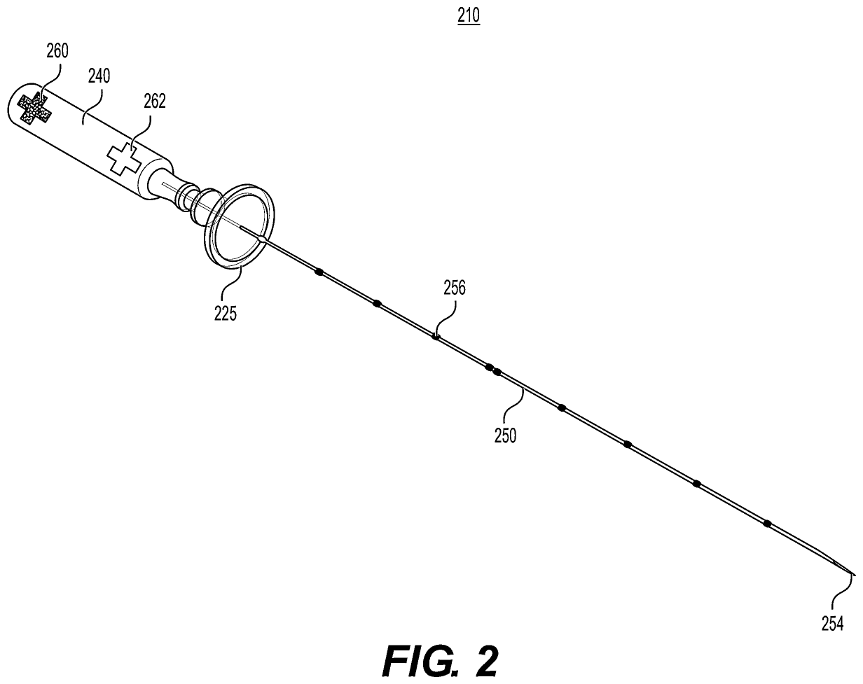

FIG. 2 shows an exemplary medical device 210 according to some aspects of the present disclosure, useful for performing a PCNL procedure, e.g., establishing the desired needle orientation and depth for accessing a kidney. Medical device 210 includes a needle 250 and a handle 240 coupled to the needle 250. Handle 240 may be generally cylinder-shaped or may have any other desired shapes, including grips with ergonomic shape for comfort of use. Handle 240 includes a body that comprises a radiolucent material, that is, a material transparent in X-ray imaging. Exemplary radiolucent materials suitable for the devices and systems herein include, but are not limited to, glass and polymers. Optionally, handle 240 may include a hub 225 proximate the needle 250.

Handle 240 includes a first marker 260 proximate a proximal end of the handle body (farthest from the needle 250) and a second marker 262 proximate a distal end of the handle body (closest to the needle 250). For example, the first marker 260 may be positioned at the proximal end of handle 240 and the second marker 262 may positioned at the distal end of handle 240. In some examples, first marker 260 may be positioned on or incorporated into a proximal face of handle 240 and second marker 262 may be positioned on or incorporated into a distal face of handle 240. In cases in which the medical device 210 includes a hub 225, the hub 225 may be at a distalmost end of the handle body. The hub 225 may be between the second marker 262 and the needle 250, or the second marker 262 may be incorporated into the hub 225.

Each of the first marker 260 and the second marker 262 may comprise a radiopaque material (e.g., barium sulfate, bismuth, tungsten, or other radiopaque metals or metal alloys). Thus, for example, the first marker 260 and the second marker 262 may be visible through the handle body, e.g., from a proximal-most end of the handle 240, through the radiolucent material, under X-ray imaging. In some aspects, the first marker 260 and the second marker 262 may be oriented perpendicular to a longitudinal axis defined by the needle 250, e.g., extending along a plane perpendicular to the longitudinal axis. Thus, for example, when viewed in the distal direction from the proximal end of the handle body, a user may be able to view both the first marker 260 and second marker 262.

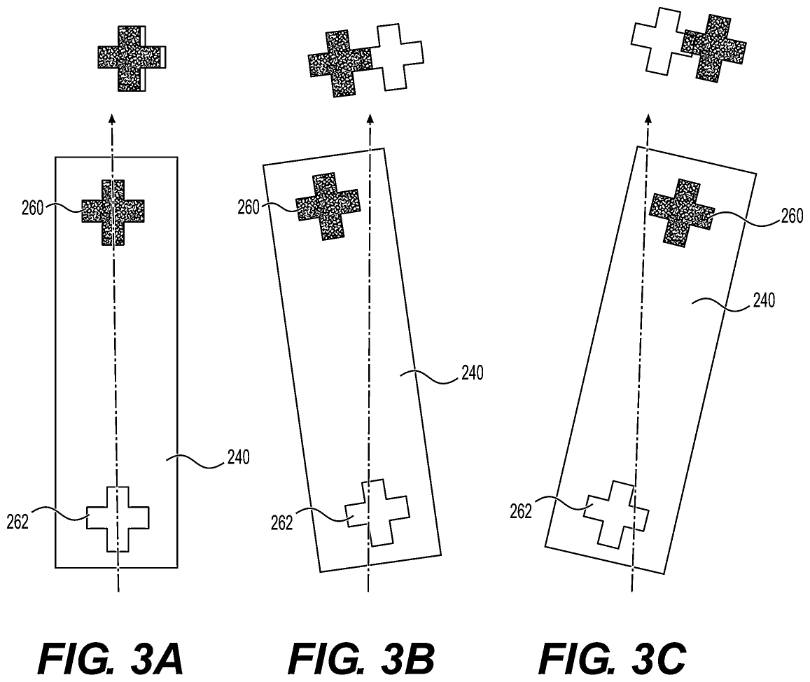

The first marker 260 may have a shape complementary to the shape of the second marker 262. For example, the first and second markers 260, 262 may have the same shape, or different shapes that together may form a complementary shape or design. The complementary shapes may assist with determining whether the markers 260, 262 are aligned with one another, e.g., to align a position of the needle 250 relative to a subject when visualized by X-ray imaging. In some examples, one of the first marker 260 or the second marker 262 is a solid shape and the other of the first marker 260 or the second marker 262 is a perimeter of the solid shape (e.g., an outline of the shape). This is illustrated, for example, in FIGS. 3A-3C.

In some examples, alignment may result in the markers 260, 262 being superimposed on each other, e.g., the first marker 260 or second marker 262 fully eclipsing the other, as seen in FIGS. 3A-3C. FIG. 3A shows alignment of the two markers 260, 262, wherein the shapes of the markers 260, 262 are superimposed on each other (in this example, the markers 260, 262 having the same shape of a plus or cross). FIGS. 3B and 3C show positions in which the markers 260, 262 are not aligned, which may indicate that the medical device 210 does not have the desired angle and orientation for needle access during a medical procedure such as PCNL.

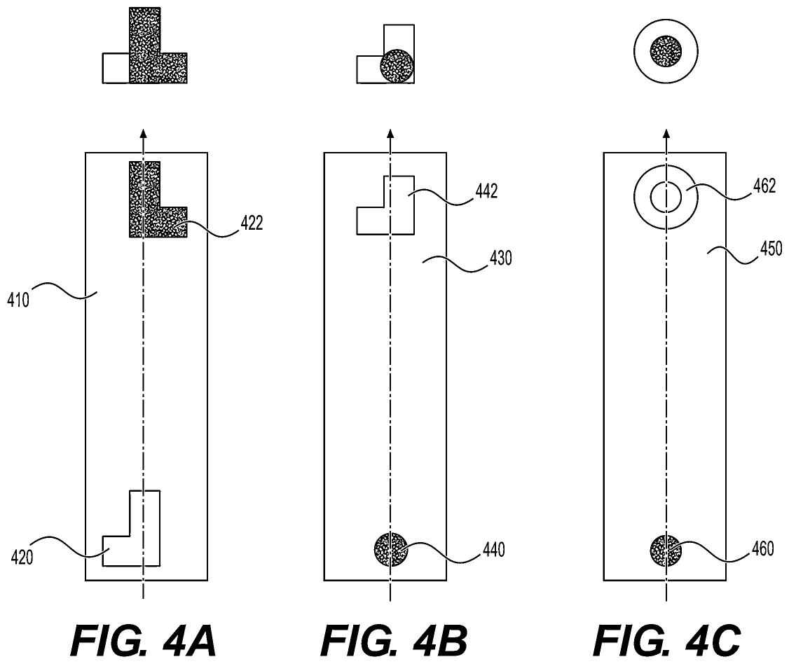

FIGS. 4A-4C show additional examples of complementary markers that may be included in the medical devices herein. For example, FIG. 4A shows two markers 420, 422 of a handle 410 of a medical device (which may include any of the features of medical device 210 above), wherein the markers 420, 422 have generally the same shape (e.g., an L shape) that when aligned, partially overlap in the central portion of each marker 420, 422. FIG. 4B shows two markers 440, 442 of a handle 430 of a medical device (which may include any of the features of medical device 210 above), wherein the first marker 420 (shown as a solid circle) completely fits within the outer perimeter of the second marker 422 (shown as the outline of an L shape) when the markers 420, 422 are aligned. That is, the circle of the first marker 420 is aligned within lower corner of the L-shape of the second marker 422. FIG. 4C shows two markers 460, 462 of a handle 450 of a medical device (which may include any of the features of medical device 210 above), wherein the first marker 460 (shown as a solid circle) completely fits within an opening of the second marker 462 (shown as a donut shape defining the opening) when the markers 420, 422 are aligned. Additional complementary shapes are contemplated herein including e.g., shapes having a mirrored orientation, L-shapes that together form a square or rectangle, hemispherical shapes that together form a circle, etc.

Referring again to FIG. 2, the needle 250 of medical device 210 may include one or more measurement marks 256, e.g., a plurality of measurement marks 256, distributed along the length of the needle 250. For example, the measurement marks 256 may be distributed at regular intervals between the handle 240 and the distal end 254 of the needle 250. The measurement marks 256 may assist a user with determining the depth of insertion of the needle 250 in a subject during a medical procedure. A narrower width between adjacent measurement marks 256 generally corresponds to higher resolution, e.g., a more precise distance measurement. The distance between measurement marks 256 may be suitable for cameras (e.g., a miniature digital camera or other imaging device used in endoscopic procedures) capable of recognizing distances of ±1 mm or ±2 mm. In some examples, the measurement marks 256 may be 1 mm apart, 2 mm apart, or 5 mm apart. The length of the needle 250 may be selected according to the type of medical procedure, e.g., the organ(s) and/or other tissues to be accessed by the needle 250. According to some aspects of the present disclosure, the needle 250 may be at least 8 cm in length, e.g., ranging from about 8 cm to about 24 cm, such as a length ranging from about 10 cm to about 20 cm, or from about 12 cm to about 18 cm. In some examples, the needle 250 may include at least two measurement marks 256, for example, a plurality of 2 to 10 measurement marks 256. Each measurement mark 256 may be a distance of at least 1 cm from an adjacent measurement mark 256, e.g., a distance ranging from about 1 cm to about 5 cm, or from about 1.5 cm to about 3.5 cm.

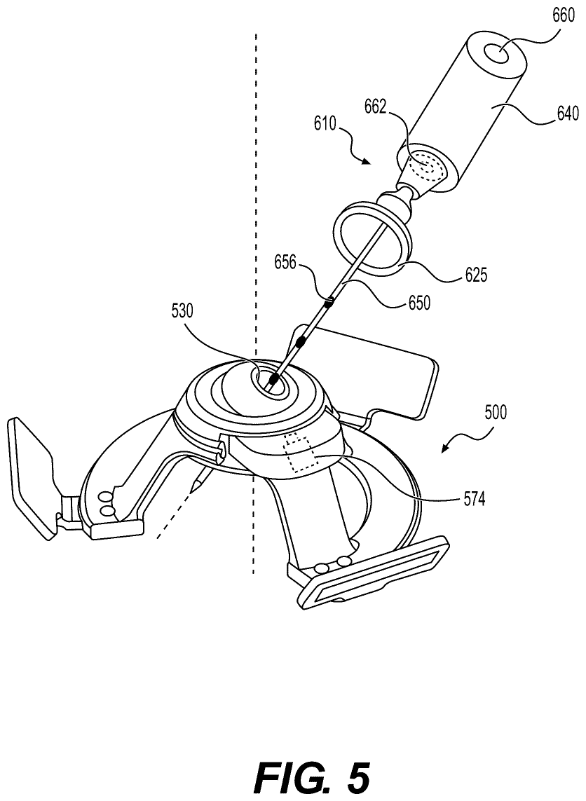

According to some aspects, the medical devices herein may be used with a fixture to assist in maintaining the position of the medical device relative to a subject and/or monitoring the needle before, during, and/or after insertion into the subject. FIG. 5 illustrates an exemplary medical system comprising a medical device 610 (which may include any of the features of medical device 210 above) and a fixture 500. Medical device 610 includes a handle 640 with radiopaque markers 660, 662, the handle 640 comprising a radiolucent material to permit observation of the markers 660, 662 under X-ray imaging. The handle 640 is coupled to a needle 650 that includes a plurality of measurement marks 656. The medical device 610 also includes a hub 625.

All or a portion of fixture 500 may comprise a radiolucent material, such as, e.g., a polymer or glass, to avoid interference with medical imaging. Fixture 500 may include one or more features for removable attachment to the skin of a subject. For example, the fixture 500 may include one or more flaps or tabs (three tabs illustrated in the example shown in FIG. 5), which may be attached to the skin with medical adhesive tape or other biocompatible adhesive suitable for temporary use.

Fixture 500 may include an opening 530 sized and shaped to receive needle 650 of the medical device 610 and to allow for movement of the needle 650 for desired angle of insertion into a subject for a medical procedure. For example, the opening 530 may allow for 180-degree rotation of the needle 650 relative to the fixture 500 (and relative to a subject). The fixture 500 may include locking features to secure the needle 650 in place once the needle 650 has the desired orientation. For example, the fixture 500 may include a lock selectively actuated by a user to maintain the angle of the needle 650 relative to the fixture 500 while allowing the needle 650 to extend and retract through the opening 530. The lock may operate by a spring mechanism or providing friction around the opening 530 to maintain the angle of the needle 650.

As mentioned above, the fixture 500 may allow for monitoring of the needle 650. For example, the fixture 500 may include a camera 574 (e.g., a miniature digital camera) coupled thereto or embedded therein. The camera 574 may face radially inward of the fixture 500 to permit observation of the needle 650 upon introduction into the opening 530. The camera 574 may function as an encoder that measures displacement of needle 650 and/or depth of the needle 650 within a subject (e.g., assisted by measurement marks 656 along the length of the needle 650). In some examples, the camera 574 may be a linear encoder. Camera 574 may be positioned to observe needle 650 as it is inserted through opening and advanced towards the skin of a subject for insertion or a medical procedure. The camera 574 may be configured to provide feedback based on sensing one or more measurement marks 656. The feedback from camera 574 may be used to determine the depth of insertion and displacement of needle 650 within the subject's body, e.g., relative to fixture 500. The camera 574 may be capable of monitoring the measurement mark(s) 656 along the needle 650 relative to the skin of the subject.

In an exemplary method using the medical devices and systems herein, a fixture (e.g., fixture 500) may be secured to the back of a subject while the subject is in a prone position. For a PCNL procedure, the fixture may be secured to the skin using a medical adhesive or adhesive tape to generally overlay the location of a kidney of the subject. This location also may lie along the path of X-ray radiation generated by a C-arm X-ray machine (e.g., between X-ray generator 110 and an image intensifier 120 shown in FIG. 1). The C-arm machine and/or the subject may be adjusted so that the X-ray beam forms an angle of 30 degrees with the subject, the X-ray beam intersecting with the target site of the subject's kidney.

A medical professional may introduce the needle of a medical device (e.g., needle 650) through an opening in the fixture (e.g., opening 530). In order to confirm accurate positioning of the needle 650 for access to the target site, the medical professional may observe whether two markers of the medical device handle (e.g., markers 660, 662) of the medical device handle are aligned with each other. Because the markers comprise a radiopaque material and the medical device handle (e.g., handle 640) comprises a radiolucent material, the markers may be readily observable by the C-arm machine. Once the medical professional confirms the markers are aligned (thereby confirming that the needle of the medical device is correctly positioned for the needle to access the kidney), the medical professional may advance the needle through the fixture opening to pierce the subject's skin. The needle may form an angle between about 25° and 35° relative to the back of the subject when inserted into the subject.

During this time, the medial professional may monitor movement of the needle by a camera of the fixture (e.g., camera 574) on a display operably coupled to the camera. By monitoring the needle with the camera, the medical professional may determine the appropriate depth of insertion of the needle to access the kidney. For example, the medical professional may determine the depth of insertion according to visibility of one or more measurement marks (e.g., measurement marks 656) along the needle within view of the camera. Accordingly, the medical professional need not adjust the position of the subject or the C-arm machine to determine the depth of insertion of the needle and proximity of the distal end of the needle to the kidney. Upon accessing the desired site within the kidney (e.g., a calyx containing a kidney stone), the medical professional may continue with treatment by aspirating urine, and breakup and removal of the stone.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a needle; and
a handle coupled to the needle, wherein the handle includes a body comprising a radiolucent material, a first marker proximate a proximal end of the body and a second marker proximate a distal end of the body, each of the first marker and the second marker comprising a radiopaque material, wherein the first marker has a shape complementary to the second marker to align a position of the needle relative to a subject when visualized by X-ray imaging.

2. The medical device of claim 1, wherein the needle includes a plurality of measurement marks distributed along a length of the needle.

3. The medical device of claim 1, the first marker and the second marker are visible from a proximal-most end of the handle through the radiolucent material.

4. The medical device of claim 1, wherein the first marker and the second marker are oriented perpendicular to a longitudinal axis defined by the needle.

5. The medical device of claim 1, wherein one of the first marker or the second marker is a shaded shape and the other of the first marker or the second marker is a perimeter of the shaded shape.

6. The medical device of claim 1, wherein the radiolucent material comprises glass or a polymer.

7. The medical device of claim 1, wherein the needle is at least 10 cm in length.

8. The medical device of claim 1, wherein the handle includes a hub at a distalmost end of the body between the second marker and the needle.

9. A medical system comprising the medical device of claim 1 and a fixture configured to receive the needle and maintain a position of the needle relative to the fixture during a medical procedure.

10. The medical system of claim 9, wherein the fixture includes a camera configured to observe the needle during the medical procedure.

11. A medical system comprising:
a medical device comprising:
a needle including a plurality of measurement marks distributed along a length of the needle; and
a handle coupled to the needle, wherein the handle includes a body comprising a radiolucent material, a first marker proximate a proximal end of the body, and a second marker proximate a distal end of the body, each of the first marker and the second marker comprising a radiopaque material; and
a fixture configured to receive the needle and maintain a position of the needle relative to a subject during a medical procedure;
wherein the first marker has a shape complementary to the second marker to align a position of the needle relative to the fixture and relative to the subject when visualized by X-ray imaging.

12. The medical system of claim 11, wherein the first marker and the second marker are visible from a proximal-most end of the handle through the radiolucent material.

13. The medical system of claim 11, wherein the fixture includes a camera configured to observe the needle during the medical procedure.

14. The medical system of claim 11, wherein the radiolucent material comprises glass or a polymer.

15. The medical system of claim 11, wherein the handle includes a hub at a distalmost end of the body, the hub being between the second marker and the needle.

16. A method of treating a subject, the method comprising:
adjusting an orientation of a medical device relative to a back of the subject, wherein the medical device comprises:
a needle; and
a handle coupled to the needle, wherein the handle includes a body comprising a radiolucent material, a first marker proximate a proximal end of the body and a second marker proximate a distal end of the body, each of the first marker and the second marker comprising a radiopaque material; and
inserting the needle into the subject to access a kidney of the subject;
wherein adjusting the orientation of the medical device includes aligning the first marker with the second marker by X-ray imaging.

17. The method of claim 16, wherein the needle is inserted while the needle forms an angle between about 25° and about 35° relative to the back of the subject.

18. The method of claim 16, wherein the radiolucent material comprises glass or a polymer.

19. The method of claim 16, wherein the needle is inserted through an opening of a fixture secured to the back of the subject, wherein the fixture maintains the orientation of the medical device relative to the subject.

20. The method of claim 19, wherein the fixture includes a camera, and the method further comprises monitoring insertion of the needle with the camera.
